(19) United States
(12) Patent Application Publication
(10) Pub. No.: US 2022/0305063 A1
(43) Pub. Date: Sep. 29, 2022

(54) PROBIOTIC COMPOSITIONS AND METHODS OF USE

(71) Applicant: New York University, New York, NY (US)

(72) Inventors: Deepak SAXENA, New York, NY (US); Xin LI, New York, NY (US)

(21) Appl. No.: 17/615,512

(22) PCT Filed: Jun. 1, 2020
(86) PCT No.: PCT/US2020/035556

§ 371 Date: Nov. 30, 2021

Related U.S. Application Data
(60) Provisional application No. 62/854,781, filed on May 30, 2019.

(51) Int. Cl. A61K 35/745; A61K 35/747; A23L 33/135; A61P 35/00; A61P 3/10; A61P 31/04; A61K 9/16

(52) U.S. Cl. CPC A61K 35/745; A61K 35/747; A23L 33/135; A61P 35/00; A61P 3/10; A61P 31/04; A61K 9/16; A61K 2035/115

(57) ABSTRACT

Provided are composition and methods for improving gut microbiome to improve pancreatic function and oral health. The probiotic composition may be administered following depletion of the existing microbiome. The probiotic compositions and methods may be used in the treatment of pancreatic malfunction as in pancreatic cancer or diabetes, or may be applied directly to the oral cavity to improve oral health.

### CROSS-REFERENCE TO RELATED APPLICATIONS

This application claims priority to U.S. Provisional patent application no. 62/854,781, filed on May 30, 2019, the disclosure of which is incorporated herein by reference.

### BACKGROUND OF THE DISCLOSURE

Human disease may be associated with dysbiosis in gut microbiome. The human microbiota, especially the gut microbiota, has even been considered to be an “essential organ”, carrying approximately 150 times more genes than are found in the entire human genome. Important advances have shown that the gut microbiota is involved in basic human biological processes, including modulating the metabolic phenotype, regulating epithelial development, and influencing innate immunity. The microbiota can carry out multiple metabolic activities ranging from catabolism and bioconversion of complex molecules to synthesis of a wide range of compounds that can affect both the microbiota and the host. In some cases the microbiota can augment pathways that are present in the host, but in others the microbiota encodes for pathways that are unique to the microbial component of the microbiome. Over the past few decades, scientists have linked the gut's composition of microbes to dozens of seemingly unrelated conditions—from diabetes to depression to obesity. Cancer has some provocative connections as well: inflammation is a contributing factor to some tumors and a few types of cancer have infectious origins. But with the explosive growth of a new class of drugs—cancer immunotherapies—scientists have been taking a closer look at how the gut microbiome might interact with treatment (e.g., immune checkpoint blockade), and how these interactions might be harnessed.

Further, diet is a modulator of the composition and the function of the gut microbiota and metabolites derived from gut microbiota processing of dietary compounds are important factors influencing host physiology. Studies have shown that low diversity in the gut microbiome associates with obesity and a higher prevalence of insulin resistance, non-alcoholic fatty liver disease (NAFLD), and low-grade inflammation. Low-grade inflammation of visceral adipose tissue may provide a link between obesity and insulin resistance. Type 2 diabetes mellitus affects more than four hundred million people around the world. It is the most common form of diabetes present in individuals and one of the leading causes of death. The gastrointestinal tract harbors a diverse ecosystem of microbes that carry out a critical role in health and disease. Recent studies have shown that microbial dysbiosis can lead to an increase in harmful metabolites that may alter systemic pathways including but not limited to insulin resistance and glucose tolerance.

However, the relationship of microbiota and specific diseased conditions is not known and there is a continued need for investigation of these relationships and approaches to overcome the challenges, such as in the area of pancreatic function.

### SUMMARY OF THE DISCLOSURE

The present disclosure provides probiotic compositions and methods of improving gut microbiome using the probiotic compositions. The present compositions and methods can be used in the treatment of conditions, such as conditions involved altered pancreatic function.

In an aspect, the present disclosure provides probiotic compositions comprising bacteria from Lactobacillus species, Akkermansia species, Ruminococcus species, Ochrobactrum species, Streptococcus species, Bifidobacterium species, non-pathogenic Escherichia coli, Bifidobacterium species, Verrucomicrobia phylum, and Firmicutes phylum. In an embodiment, the probiotic composition does not contain any Escherichia coli species. In an embodiment, the composition has one or more of Lactobacillus species, Akkermansia species, Ruminococcus species, Streptococcus species, Verrucomicrobia class/species, and Firmicutes class/species. In an embodiment, the probiotic composition comprises or consists essentially of two or more bacteria selected from: one or more Lactobacillus species, Akkermansia species, Ruminococcus species, Ochrobactrum species, Streptococcus species, Bifidobacterium species, Escherichia coli, Bifidobacterium species, Verrucomicrobia species, and Firmicutes species. In an embodiment, the probiotic composition comprises or consists essentially of lactobacillus and streptococcus species.

The compositions can be used for improvement of pancreatic function and/or for improvement of oral health. The method of the present disclosure comprises administering to a subject in need of treatment a probiotic composition disclosed herein. In an embodiment, prior to administration of the probiotic composition, the existing microbiome in the individual is depleted, such as by administration of certain antibiotics, such as vancomycin, neomycin, metronidazole, and amphotericin, or combinations thereof.

### BRIEF DESCRIPTION OF THE DRAWINGS

FIG. 1 shows that repopulation of antibiotic-treated KC mice with the pancreatic ductal adenocarcinoma (PDA) microbiome reverses tumor-protection. KC mice treated with an ablative oral antibiotic regimen for 8 weeks were repopulated with i) feces from 3-month old WT mice, ii) feces from 3 month-old KPC mice, or iii) sham-repopulated (vehicle only). Mice were sacrificed 8 weeks later and pancreas weights from each cohort were compared to each other and to age-matched control KC mice that were not treated with antibiotics. Ablative antibiotics slowed tumor growth however, repopulation with the KPC mouse derived feces (but not WT) reversed the tumor protection. This experiment was repeated three times.

FIG. 2. Heat-map of top at genus level showing longitudinal gut microbial diversity in MKR and WT mice (n=3/group). Double hierarchical linkage clustering of the cohorts was based on composition (y-axis) and abundance (x-axis) of gut microbiota. Average abundances of each genus are row normalized and are indicated by the gradient. The dendrogram on the x-axis indicates the distinct clusters of each cohort.

FIG. 3. Scheme for design to test probiotics. All experimental mice received antibiotics treatment at 9-week-old to become bacterial depleted prior treatment with sham, probiotic or fecal bacterial transferring by oral gavage weekly for indicated time period. Fecal and tail snipping blood samples were collected at indicated time points.

FIG. 4. Results for glucose tolerance test at 12 weeks post treatment. All mice were overnight fasted and tested for fasting glucose level before intraperitoneal injection of glucose at 2 gm/kg body weight. The glucose levels were measured at 15 min, 30 min, 60 min and 120 min after injection of glucose.

FIG. 5. Bacterial H₂S production (Y-axis) as an indicator of VOC was determined by co-culturing bacteria with oral epithelial cells. Data shown are mean values for triplicate.

### DETAILED DESCRIPTION OF THE DISCLOSURE

This disclosures describes that the microbiome plays a central role in instructing the immune suppressive tumor microenvironment in conditions associated with pancreatic malfunction including pancreatic cancer and diabetes. An example of pancreatic cancer in which the microbiome plays a central role is pancreatic ductal adenocarcinoma (PDA).

In an aspect, this disclosure provides probiotic compositions comprising combinations of bacteria that can be used for treating or preventing conditions associated with pancreatic malfunction.

In an aspect, this disclosure provides methods for treating or preventing conditions associated with pancreatic malfunction. The method comprises administering to an individual in need of treatment, a probiotic composition described herein. In an embodiment, the method comprises depleting the existing gut microbiome (entirely or selectively, complete or partial) in an individual in need of treatment, and replenishing the depleted microbiome with the probiotic compositions of the present disclosure. In an embodiment, the majority, or all of gram negative microbes from the gut are depleted. The existing microbiome does not need to be completely deleted. In an embodiment, the total bacterial level in the gut is preferably reduced to 10³ to 10⁴ bacteria or lower. The term “gut” as used herein means small and large intestines. Generally there are 10⁹ to 10¹² bacteria are present in the gut.

For depleting the existing gut microbiome, one or more antibiotics may be used. The bacteria that are preferably depleted include opportunistic gram positive and/or gram negative bacteria. In an embodiment, the gut bacteria that are deleted are only gram negative bacteria and opportunistic gram positive bacteria. In an embodiment, the deleted bacteria are gram negative bacteria only or opportunistic gram positive bacteria only. Examples of antibiotics that may be used for depletion of existing gut microbiome include vancomycin, neomycin, metronidazole, and amphotericin. In an embodiment, a combination of one or more of vancomycin, neomycin, metronidazole, and amphotericin may be used. In an embodiment, all of these antibiotics may be used. In an embodiment, one or more of glycopeptide antibiotics, aminoglycoside antibiotics, quinolone antibiotics, nitroimidazoles antibiotics and antifungals may be used. In an embodiment, a combination of ciprofloxacin (belonging to the quinolone antibiotics) (such as at a dose of 500 mg PO BID days 1-29) and metronidazole (such as at a dose of 500 mg PO TID days 1-29) may be used. If more than one antibiotic is to be administered, the antibiotics may be administered together or separately, concurrently or sequentially, by same routes or different routes, using the same regimen or different regimens. Monitoring levels of bacteria to confirm depletion of gut bacteria may be carried out by qPCR and sequencing in a tissue/content sample from the gut. For example, fecal samples may be used as samples from the gut. Examples of bacteria that may be completely or partially depleted include Turicibacter, Sutterella, Odorbacter, Mucispirillum. Some desirable bacteria, such as Akkermansia may also be depleted. Repopulation of the gut with the desired bacteria may be carried out immediately after depletion (such as after cessation of the antibiotic treatment), or a suitable period after that. Repopulation may comprise a single does or multiple doses of desired bacteria. Any number of bacteria that is capable of populating the gut may be used. For example, first three weeks everyday 10⁸-10⁹ colony forming units of bacterial cells can be transferred. Repopulation may be carried out by colonoscopy, endoscopy, sigmoidoscopy, or enema. The term “colony forming unit” or cfu may be used herein interchangeably with the number of bacteria. A CFU is a measure of viable bacterial cells.

In an embodiment, the probiotic compositions are termed herein as POC518 and POC519. The probiotic compositions can be used to modulate gut microbiome to: 1) enhance cancer treatment and/or 2) increase glucose tolerance in diabetic patients, and/or 3) improve oral health and/or 4) treat halitosis, and other effects.

The present disclosure can be used to develop microbiome based therapeutics to enhance immunotherapy treatment of cancer. This approach may provide much needed boost to various immunotherapies.

This disclosure can also be used for modulating gut microbiome using the present formulations to achieve better glucose tolerance. For example, the formulations can be used in diabetic population to control blood glucose.

Probiotics refer to microorganisms that are considered to provide health benefits when administered to a subject. The probiotic compositions of the present disclosure include bacteria from one or more of the following—Lactobacillus species, Akkermansia species, Ruminococcus species, Ochrobactrum species, Streptococcus species, Bifidobacterium species, Escherichia coli, Bifidobacterium species, Verrucomicrobia species, and Firmicutes species. In an embodiment, the probiotic composition does not contain any Escherichia coli species. In an embodiment, the only E. coli in the probiotic composition is E. coli Nissel 1917. In an embodiment, the composition has one or more of Lactobacillus species, Akkermansia species, Ruminococcus species, Streptococcus species, Verrucomicrobia species, and Firmicutes species. In an embodiment, the probiotic composition comprises or consists essentially of two or more bacteria selected from: one or more Lactobacillus species, Akkermansia species, Ruminococcus species, Ochrobactrum species, Streptococcus species, Bifidobacterium species, Escherichia coli, Bifidobacterium species, Verrucomicrobia species, and Firmicutes species. In an embodiment, the probiotic composition comprises or consists essentially of lactobacillus and streptococcus species, particularly for oral health use. In an embodiment, the probiotic composition comprises or consists essentially of Lactobacillus species, Akkermansia species, Ruminococcus species, Ochrobactrum species, Streptococcus species, Bifidobacterium species, non-pathogenic Escherichia coli, Bifidobacterium species, Verrucomicrobia species, and Firmicutes species

Two examples of probiotic compositions, referred to herein as POC518 and POC519, are provided below.

Bacterial strains in POC518.
(1) Lactobacillus acidophilus,
(2) L. casei, L. paracasei,
(3) L. reuteri,
(4) Akkermansia muciniphila,
(5) Ruminococcus bromii,
(6) Streptococcus thermophilus and Bifidobacterium breve,
(7) Escherichia coli Nissle 1917,
(8) Bifidobacterium lactis, and
(9) A plurality of classes, such as three belonging to phylum Verrucomicrobia and Firmicutes.

Bacterial strains in POC519:
(1) Lactobacillus acidophilus,
(2) L. casei,
(3) L. reuteri,
(4) Akkermansia muciniphila,
(5) Ruminococcus bromii,
(6) Streptococcus salivarius, and
(7) 3 classes/genuses belongs to phylum Verrucomicrobia and Firmicutes.

In an embodiment, the probiotic formulation may be represented as comprising bacteria comprising, consisting essentially of or consisting of:
(1) Lactobacillus acidophilus,
(2) L. casei, or L. paracasei,
(3) L. reuteri,
(4) Akkermansia muciniphila,
(5) Ruminococcus bromii,
(6) Streptococcus thermophiles
(7) Bifidobacterium breve,
(8) Escherichia coli Nissle 1917,
(9) Bifidobacterium lactis,
(10) Verricomicrobia verrucomicrobiae, and
(11) Firmicutes bacilli

In an embodiment, the probiotic formulation may be represented as comprising bacteria comprising, consisting essentially of or consisting of:
(1) Lactobacillus acidophilus,
(2) L. casei,
(3) L. reuteri,
(4) Akkermansia muciniphila,
(5) Ruminococcus bromii,
(6) Streptococcus salivarius
(7) Verricomicrobia Verrucomicrobiae, and
(8) Firmicutes Bacilli

The bacteria may be isolated from animal or human fecal samples or may be obtained from a commercial source, such as American Tissue Type Collection (ATCC). To preapare the probiotic each bacteria type may be grown separately (such as by inoculation in appropriate broth etc.). Following growth, bacteria may be isolated from the culture media, and bacteria may be lyophilized separately or may be combined in the desired amounts for a probiotic use. The lyophilized probiotic material may be used for repopulation of the gut microbiome.

These bacteria may be present in amounts sufficient to repopulate the gut. For example, in any of the formulations describes herein, the number of bacteria administered in the present probiotic formulations can be (CFUs of each) Lactobacillus acidophilus from 10⁸ to 10¹⁰, L. casei and/or L. paracasei (together) from 10⁸ to 10¹⁰, L. reuteri from 10⁸ to 10¹⁰, Akkermansia muciniphila from 10⁷ to 10⁹, Ruminococcus bromii from 10⁷ to 10⁹, Streptococcus thermophiles or Bifidobacterium breve (together) from 10⁶ to 10⁸, Escherichia coli Nissle 1917 from 10⁸ to 10¹⁰, Bifidobacterium lactis from 10⁸ to 10⁹. Verricomicrobia (such as verrucomicrobiae), and/or Firmicutes (such as bacilli) (together) from 10⁶ to 10⁷. The CFUs in the probiotics are provided as per dose. Variations of the amount of CFUs can be made so long as repopulation of the gut is achieved.

In an embodiment, the number of bacteria administered in the present probiotic formulations can be 10⁸ CFUs of Lactobacillus acidophilus, 10⁸ CFUs of L. casei and/or L. paracasei (together), 10⁸ CFUs of L. reuteri, 10⁷ CFUs of Akkermansia muciniphila, 10⁷ CFUs of Ruminococcus bromii, 10⁶ CFUs of Streptococcus thermophiles or Bifidobacterium breve (together), 10⁸ CFUs of Escherichia coli Nissle 1917, 10⁸ CFUs of Bifidobacterium lactis, and 10⁶ CFUs of Verricomicrobia (such as verrucomicrobiae), and/or Firmicutes (such as bacilli) (together). The CFUs in the probiotics are provided as per dose. Variations of the amount of CFUs can be made so long as repopulation of the gut is achieved.

In one embodiment, the disclosure provides a probiotic composition comprising, consisting essentially of, or consisting of bacteria of the genus Lactobacillus, Akkermansia, Ruminococcus, Streptococcus, Verrucomicrobia, and Firmicutes. In an embodiment, the probiotic may further comprise bacteria from one or more of the genuses Bifidobacterium, and/or Escherichia. In an embodiment, the only bacteria present in the probiotic belong to the genuses Lactobacillus, Akkermansia, Ruminococcus, Streptococcus, Verrucomicrobia, and Firmicutes. In an embodiment, the only bacteria present in the probiotic belong to the genuses Lactobacillus, Akkermansia, Ruminococcus, Streptococcus, Verrucomicrobia, and Firmicutes, Bifidobacterium, Escherichia, and Bifidobacterium.

In an embodiment, the disclosure provides a probiotic composition comprising, consisting essentially of, or consisting of the following bacteria: Lactobacillus acidophilus; L. casei, L. reuteri; Akkermansia muciniphila, Ruminococcus bromii; and strains of phylum Verrucomicrobia and phylum Firmicutes, and optionally, may contain one or more of Streptococcus thermophiles, Bifidobacterium breve, Escherichia coli Nissle 1917, Bifidobacterium lactis, and Streptococcus salivarius.

The amount of bacteria (individual type or all types) per dose may be 100 million to 1 billion bacterial cells (i.e., CFUs) and all values and ranges therebetween. In an embodiment, a dose may have more than 1 billion bacteria (individual type or all types). A dose may be a tablet, capsule, or a specified amount of the formulation in any form. In various embodiments, the bacteria (individual type or all types) per dose may be 100, 200, 300, 400, 500, 600, 700, 800, 900 million or 1 billion, 2 billion, 3, billion etc. The probiotics can be administered after depletion of the endogenous gut microbiome (e.g., by administration of antibiotics) after a suitable period of time. For example, the probiotics may begin to be administered 2 weeks after the antibiotics or after 3, 4, 5 or 6 weeks after depletion. In an embodiment, the probiotics may be administered 4-6 weeks or 6-8 weeks after cessation of the antibiotic regimen or after depletion of existing gut microbiome as described herein, and may be continued as necessary to repopulate the gut.

The composition can be formulated for oral administration. The present oral compositions may be in the form of a chewable formulation, a dissolving or dissolved formulation, an encapsulated/coated formulation, a multi-layered lozenges (to separate active ingredients and/or active ingredients and excipients), a slow release/timed release formulation, or other forms suitable for oral delivery known in the art. It may be in the form of a tablet, lozenges, pill, capsule, drops, paste or the like. The formulations may also be present as encapsulated or incorporated into micelles, liposomes, cyclodextrins, polymers and the like.

The probiotic formulations, including pediatric formulations, may be flavored (e.g. fruit flavored, such as cherry, strawberry, blueberry etc.) and may be in a variety of shapes or colors.

In an aspect, the present disclosure provides pharmaceutically acceptable compositions which comprise a therapeutically-effective amount of one or more of the bacterial species as described above, the cell therapies as described above, and/or the cytokines as discussed above, formulated together with one or more pharmaceutically acceptable excipients. In an aspect, the present invention provides pharmaceutically acceptable compositions which comprise a therapeutically-effective amount of one or more of the bacteria species as described above, formulated together with one or more pharmaceutically acceptable excipients and other therapeutically effective medications known in the art allowing for but not limited to combination therapies to improve overall efficacy of each individual therapeutic or to limit the concentration of either therapeutic to avoid side effects and maintain efficacy. The active ingredient and excipient(s) may be formulated into compositions and dosage forms according to methods known in the art. The pharmaceutical compositions of the present invention may be specially formulated for administration in solid or liquid form, including those adapted for the following: oral administration, for example, tablets, capsules, powders, granules, and aqueous or non-aqueous solutions or suspensions, drenches, or syrups, frozen or freeze-dried forms; or intrarectally, for example, as a pessary, cream or foam. The probiotic compositions may be present in a lyophilized form (i.e., freeze-dried form). The bacteria may be lyophilized individually or the entire probiotic composition may be lyophilized.

A therapeutically effective amount of the pharmaceutical composition of the present invention is sufficient to promote the health of the intestines, or to treat or prevent a disease characterized by abnormality of pancreatic function. For example, the present compositions may be used to treat cancers of the pancreas, such as PDA, or glucose imbalance conditions, such as diabetes, oral health diseases, and halitosis. The dosage of active ingredient(s) may vary, depending on the reason for use and the individual subject. The dosage may be adjusted based on the subject's weight, the age and health of the subject.

The term “therapeutically effective amount” as used herein refers to an amount of an agent sufficient to achieve, in a single or multiple doses, the intended purpose of treatment. Treatment does not have to lead to complete cure, although it may. Treatment can mean alleviation of one or more of the symptoms or markers of the indication. The exact amount desired or required will vary depending on the particular compound or composition used, its mode of administration, patient specifics and the like. Appropriate effective amount can be determined by one of ordinary skill in the art informed by the instant disclosure using only routine experimentation. Within the meaning of the disclosure, “treatment” also includes relapse, or prophylaxis as well as the alleviation of acute or chronic signs, symptoms and/or malfunctions associated with the indication. Treatment can be orientated symptomatically, for example, to suppress symptoms. It can be effected over a short period, over a medium term, or can be a long-term treatment, such as, for example within the context of a maintenance therapy. Administrations may be intermittent, periodic, or continuous.

The phrase “pharmaceutically acceptable” is employed herein to refer to those compounds, materials, compositions, and/or dosage forms which are suitable for use in contact with the tissues of the subject with toxicity, irritation, allergic response, or other problems or complications, commensurate with a reasonable benefit/risk ratio.

The phrase “pharmaceutically-acceptable excipient” as used herein refers to a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject. Each excipient should be “acceptable” in the sense of being compatible with the other ingredients of the formulation and not injurious to the subject. Some examples of materials which can serve as pharmaceutically-acceptable excipients include sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

Excipients are added to the composition for a variety of purposes. Diluents increase the bulk of a solid pharmaceutical composition, and may make a pharmaceutical dosage form containing the composition easier for the patient and caregiver to handle. Diluents for solid compositions include, for example, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. EUDRAGIT®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch.

In liquid pharmaceutical compositions of the present invention, the bacterial species and any other solid excipients are dissolved or suspended in a liquid carrier such as water, water-for-injection, vegetable oil, alcohol, polyethylene glycol, propylene glycol or glycerin. Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol. Liquid pharmaceutical compositions of the present invention may also contain a viscosity enhancing agent to improve the mouth feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum. A liquid composition may also contain a buffer such as gluconic acid, lactic acid, citric acid or acetic acid, sodium gluconate, sodium lactate, sodium citrate or sodium acetate.

Sweetening agents such as sorbitol, saccharin, sodium saccharin, sucrose, aspartame, fructose, mannitol and invert sugar may be added to improve the taste. Flavoring agents and flavor enhancers may make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid. Preservatives and chelating agents such as alcohol, sodium benzoate, butylated hydroxy toluene, butylated hydroxyanisole and ethylenediamine tetraacetic acid may be added at levels safe for ingestion to improve storage stability.

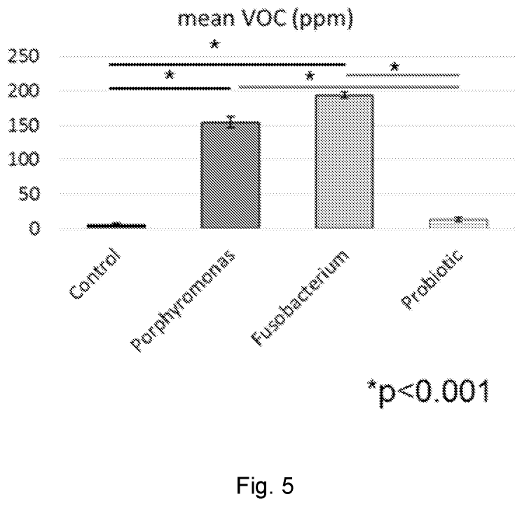

The present compositions can be used to treat conditions including pancreatic cancer, diabetes, oral health conditions including halitosis. The compositions can also be used to improve immune response, oral hygiene, including bone density and treatment of bad breath. Halitosis is also a characteristic symptom of periodontal disease, and is caused by the production of volatile sulphur compounds (VSCs), such as hydrogen sulphide (H2S) and methyl mercaptan, by sulfate-reducing bacteria. The major cultivatable periodontal opportunistic pathogens, Porphyromonas gingivalis (Pg), Fusobacterium nucleatum (Fn) and Tannerella forsythia (Tf), are reported to have produced H₂S in an in vitro system as measured by gas chromatography. These pathogens colonize the surface of tongue play significant role in H₂S production. In 80 to 90 percent halitosis cases involve bacteria from the oral cavity. Here we showed in vitro cell culture model and POC519 bacterial formulation to reduce H₂S production (FIG. 5).

The subject may be any animal, including human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

The subjects, such as human subjects may be healthy or may be suffering from or at risk for a disease or condition associated with abnormal functioning of the pancreas. The subject is generally diagnosed with the condition of the subject invention by skilled artisans, such as a medical practitioner.

The present probiotic compositions and methods may be used in conjunction with fecal matter transplant therapies (FMT), which involves using intestinal bacteria from a healthy individual's fecal matter and then processing and transferring that bacteria to the infected patient directly. The fecal matter may be processed to extract the bacteria. The transplantation of the fecal matter is generally carried out by colonoscopy, endoscopy, sigmoidoscopy, or enema. FMT may also be carried out by using frozen or freeze-dried fecal microbiota administered in pill form.

The appropriate dosage and treatment regimen of the probiotic compositions may be determined or recommended by a clinician or nutritionist. In general, one or more doses may be administered per day for a day, week, and month or longer if needed. For example, a dose may be administered every day for 1 week.

Some specific embodiments of the present disclosure are provided below.

A lyophilized probiotic composition comprising, consisting essentially of, or consisting of two or more, or all of: Lactobacillus acidophilus, L. casei, and/or L. Paracasei, L. reuteri, Akkermansia muciniphila, Ruminococcus bromii, Streptococcus thermophilus and/or Bifidobacterium breve, Escherichia coli Nissle 1917, Bifidobacterium lactis, and strains of phylum Verrucomicrobia and Firmicutes.

A lyophilized probiotic composition comprising, consisting essentially of, or consisting of two or more, or all of: Lactobacillus acidophilus, L. casei, L. reuteri, Akkermansia muciniphila, Ruminococcus bromii, Streptococcus salivarius, and strains of phylum Verrucomicrobia and Firmicutes.

A lyophilized probiotic composition comprising, consisting essentially of or consisting of two or more, or all of: Lactobacillus acidophilus (10⁸ CFUs), L. casei, and/or L. Paracasei (10⁸ CFUs), L. reuteri (10⁸ CFUs), Akkermansia muciniphila (10⁷ CFUs), Ruminococcus bromii (10⁷ CFUs), Streptococcus thermophilus and/or Bifidobacterium breve (10⁶ CFUs), Escherichia coli Nissle 1917 (10⁸ CFUs), Bifidobacterium lactis, and strains of phylum Verrucomicrobia and Firmicutes (10⁶ CFUs).

A lyophilized probiotic composition comprising, consisting essentially of or consisting of two or more or all of: Lactobacillus acidophilus (10⁸ CFUs); L. casei (10⁸ CFUs); L. reuteri (10⁸ CFUs); Akkermansia muciniphila (10⁷ CFUs); Ruminococcus bromii(10⁷ CFUs); Streptococcus salivarius (10⁹ CFUs); and strains of phylum Verrucomicrobia and Firmicutes (10⁶ CFUs).

A method for improving pancreatic function comprising administering to an individual in need of treatment (such as an individual with impaired pancreatic function) one or more antibiotics to deplete existing gut microbiome (such as to a level of 10³ or 10⁴), and administering a probiotic composition of any of the embodiments described herein, such as POC518 or POC519, in amounts such that the gut microbiome is repopulated. The pancreatic condition that is being treated may be pancreatic cancer, such as pancreatic ductal adenocarcinoma, or diabetes, such as type 2 diabetes. The antibiotics may be a cocktail of antibiotics comprising two or more of vancomycin, neomycin, metronidazole and amphotericin.

In an embodiment, the present probiotics may be administered to an individual who has depleted gut microbiome or may be administered to an individual without depleting their gut microbiome with antibiotics.

In an embodiment, the present probiotic compositions may be used in the treatment of halitosis. For example, POC519 may be used directly in the oral cavity for the treatment of halitosis. The probiotic may be used by itself in the form of a rinse, paste, liquid, gel or chewable or other tablets, or may be incorporated into toothpastes, other rinses (such as dental or oral mouthwashes), oral or dental appliances, or any other device that may come in contact with the oral cavity.

The following examples are provided to further illustrate the invention and are not intended to be limiting.

### Example 1

Bacterial strains were isolated from animal or human samples and sequenced, identified and stored at −80° C. until used or were obtained from ATCC. To prepare the probiotic bacterial cocktail, each bacterial strain was individually inoculated into a broth and incubated at 37° C. for 48-72 h. Cell suspensions was transferred to 50 ml sterile tubes under aseptic conditions and centrifuged at 4000 g for 10 min. The supernatant was discarded, and the cultured cells was washed twice using phosphate buffered saline (PBS). The suspension containing bacteria cells (10⁸-10⁹ CFU/ml) were directly added to the carboxymethylcellulose sodium (CMC) solution. CMC solution (1% w/v) for lyophilization was prepared by the gradual addition of 1 g CMC powder to 100 ml distilled water at 70° C. The solution was mixed well using a magnetic stirrer at 500 rpm for 40 min to ensure uniform dispersion. When the solution temperature had cooled to 37° C., bacteria was added to the solution to reach a final concentration of 10⁹ CFU/ml. The solution was lyophilized using a freeze dryer (FREEZONE 2.5 Liter Benchtop Freeze Dryer). The viability of bacterial cells in the formulation was determined by suspending 1 g of lyophilized powder in 1 ml of the sterile PBS. The solution was vortexed for 30s, and an appropriate dilution series was prepared. Enumeration of the bacteria on agar plates was carried out in triplicate using standard colony count technique. Repopulation was performed by gastric gavage. 16S sequencing and qPCR was used to determine the colonization of probiotic bacteria in the gut.

### Example 2

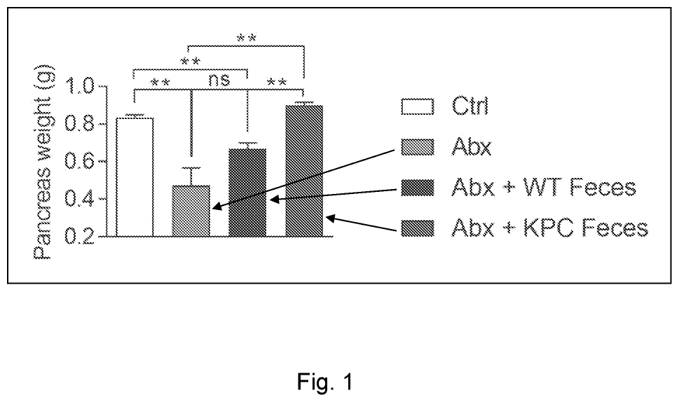

We found that the cancerous pancreas harbors a markedly more abundant microbiome compared to normal pancreas in mice and humans. Further, we found that ablation of the microbiome in mice protected against pre-invasive and invasive PDA. Conversely, transfer of bacteria from PDA-bearing hosts, but not controls, reversed this tumor-protection. We showed that the microbiome exerts potent suppressive influences on the inflammatory tumor microenvironment. Specifically, the microbiome collectively sets the tolerogenic inflammatory program in PDA promoting the recruitment of myeloid-derived suppressor cells (MDSC) and M2-like macrophages, driving Th2 and Treg differentiation of CD4+ T cells and suppression of CD8+ T cells. Further, we showed that ablating pathogenic bacteria upregulated PD-1 expression on T cells and enabled efficacy for checkpoint-based immunotherapy. Our data (FIG. 1) indicates the microbiome can be used as a therapeutic target in both the modulation of disease progression and in improving the efficacy of immunotherapy.

KC mice, which develop spontaneous pancreatic neoplasia by targeted expression of mutant Kras in the pancreas. C57BL/6 (H-2Kb) mice (WT) were originally purchased from The Jackson Laboratory and were bred in-house and crossed with the KC model after 8 generations. KPC mice express mutant intrapancreatic Kras and Trp53. Littermate controls were used in experiments. Animals were housed in a specific pathogen-free vivarium and fed standard mouse chow. To ablate the gut microbiome, 6-week-old WT or KC mice were administered an antibiotic cocktail by oral gavage daily for five consecutive days. Controls were gavaged with PBS. The oral gavage cocktail contained vancomycin (50 mg/mL; Sigma), neomycin (10 mg/mL; Sigma), metronidazole (100 mg/mL; Santa Cruz Biotech), and amphotericin (1 mg/mL; MP Biomedicals). Additionally, for the duration of the experiments, mouse drinking water was mixed with ampicillin (1 mg/mL; Santa Cruz Biotech), vancomycin (0.5 mg/mL; Sigma), neomycin (0.5 mg/mL; Sigma), metronidazole (1 mg/mL; Santa Cruz Biotech), and amphotericin (0.5 μg/mL; MP Biomedicals). In fecal transfer experiments, six fecal pellets from mice were collected and resuspended in 1 mL of PBS, and 200 μL of the fecal slurry was used for orogastric gavage every other day for 2 weeks.

### Example 3

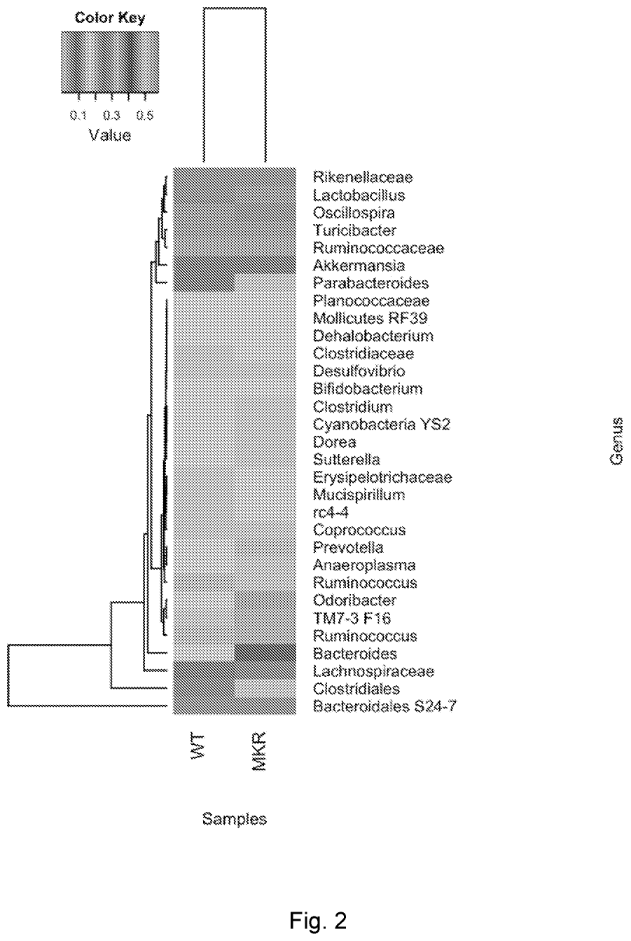

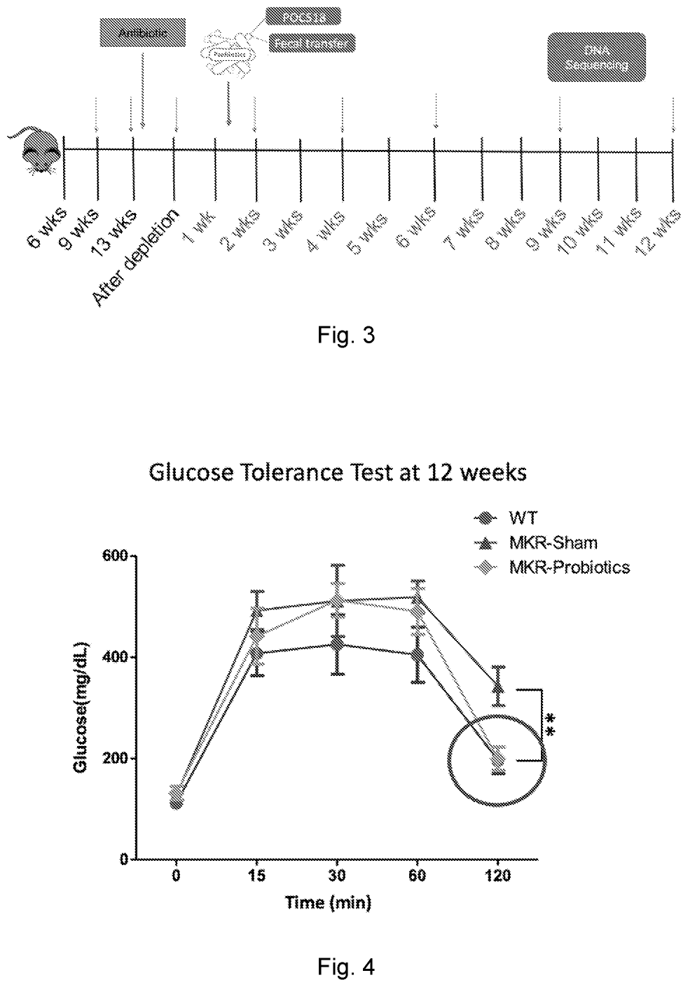

This example describes modulating gut microbiome with the probiotics of this disclosure to result in better glucose tolerance. The gut microbiome plays an important role in T2DM metabolic disorder and presents a potential target for bio-therapeutic treatments. Our preliminary data on 16S rRNA using MiSeq and mouse fecal samples as a proof-of-principal to determine whether the microbiome in our WT and MKR (Muscle IGF-I receptor (IGF-IR)-lysine-arginine) mice model was altered. Heat map analysis indicated that bacterial communities in the T2DM and WT mice (age and gender matched littermate (n=3/group)) were different and formed two separate clusters indicative of colonization of T2DM mice with a distinct microbiome with progressive hyperglycemia. There were marked increases in the prevalence of phyla Actinobacteria, Deferrlbacteres, Tenericutes and TM7 in T2DM group. At the genus level, Bacteroides, Ruminococcus, Parabacteroides, Prevotella, Oscillospira, Ruminococcus, Rickenellaceae, Lachnospiraceae, and Clostridiales significantly (p<0.05) increased in T2DM mice suggesting microbial dysbiosis in this group (FIG. 2). Evidence of the specific phyla involved in gut microbiome dysbiosis and probiotic effects in T2DM may provide new insights regarding its pathophysiological relevance and pave the way for new therapeutic approaches.

The study was carried out as follows. POC518 was used in this experiment. 3 cohorts were used—wild type (3), MKR sham (4), and MKR Probiotic (4). This was a 27 week study. Antibiotic administered via oral gavage for two weeks (every other day in the first week) and maintained in drinking water (during week second week) to both MKR cohorts at age thirteen weeks. Probiotics (10⁹ CFU/mL) were administered to MKR cohorts: MKR Sham (fecal transfer) and MKR Pro (PCO518) by oral gavage beginning at week 16, 2× per week for 12 weeks. Fecal and blood samples were collected under sterile conditions weekly. Glucose was measured via blood samples collected from the tail tip using Bayer Contour blood glucose monitor system. DNA extraction, PCR 16S rRNA amplification of V3-V4 hypervariable region, DNA analysis of 16S rRNA gene via Illumina MiSeq generated sequence data, and finally data analysis was carried out by QIMME 1.9.1 and R.

The results indicate that after challenge with either probiotic consortium or from MKR fecal transfer, the MKR_Pro group was able to maintain the glucose level of healthy WT control (non-diabetic) while MKR_Sham levels remained consistent with baseline measures (FIG. 4). As such, altering the gut microbiome using POC518 or POC519 can lead to improved glucose tolerance.

### Example 4

Halitosis, bad breath or oral malodor are all synonyms for the same pathology. Halitosis has a large social and economic impact. For the majority of patients suffering from bad breath, it causes embarrassment and affects their social communication and life. Moreover, halitosis can be indicative of underlying diseases. Halitosis is also a characteristic symptom of periodontal disease, and is caused by the production of volatile Sulphur compounds (VSCs), such as hydrogen sulphide (H₂S) and methyl mercaptan, by sulfate-reducing bacteria. Importantly, the major cultivatable periodontal opportunistic pathogens, Porphyromonas gingivalis (Pg), Fusobacterium nucleatum (Fn) and Tannerella forsythia (Tf), are reported to have produced H₂S in an in vitro system as measured by gas chromatography. These pathogens colonize the surface of tongue play significant role in H₂S production. In 80 to 90 percent halitosis cases involve bacteria from the oral cavity. Here we used in vitro cell culture model and POC519 bacterial formulation to determine whether cocktail of certain bacteria can reduce H₂S production.

Methods

Measurement of hydrogen sulfide from bacteria.

We used two major bacteria Porphyromonas spp and Fusobacterium spp involved in halitosis and periodontal diseases, and probiotic cocktail (POC519). Bacteria were cultured in broth medium until they reached late log growth phase, and the concentration of all strains was then adjusted to 10⁸-10⁹ cell/ml. Subsequently, the bacterial suspension was used for detecting H₂S production in bacterial biofilm culture and in presence of oral epithelial cells.

Calorimetric method: The bismuth sulfide method was modified to by using a 5 mM concentration of bismuth(III)chloride. Bacteria were diluted in peptone solution to 10⁹ cells/mL. Aliquots (100 ml) of the bacterial suspension were mixed with an equal amount of newly prepared bismuth solution (0.4 M triethanolamineHCl, pH 8.0; 10 mM bismuth(III)chloride; 20 mM pyridoxal 5-phosphate monohydrate, 20 mM EDTA and 40 mM L-cysteine) in microtiter plates. H₂S production was monitored by detecting black B S is precipitated. Intensity of black precipitate was visually scaled, from no color production (0) to maximum black color production after 24 h.

H₂S production in epithelial cell and bacterial co-culture model: Human oral epithelial origin cell line OKF6 was used in this study. The cells were cultured in Keratinocyte-Serum Free Medium supplemented with 50 μg/ml bovine pituitary extract, 5 ng/ml epidermal growth factor in humidified atmosphere of 5% CO2 at 37° C. The bacterial species tested for H₂S-producing capacity were Porphyromonas spp, Fusobacterium spp, and probiotic cocktail (POC519). The species were grown on appropriate agar plates under optimal conditions. Desired concentration of theses bacteria or POC519 (10⁸ cfu/ml) was added to 2M epithelial cells in 25 ml Corning Primaria Tissue Culture Flasks and incubated for 24 hrs as mentioned before. Handheld Hydrogen Sulfide (H₂S) Gas Detector with range from 0 to 500 ppm was used for H₂S production.

Results:

In calorimetric method bacterial hydrogen sulfide (H₂S) production from cysteine measured with colorimetric methods in microtiter plate format, recorded as black bismuth sulfide precipitation formation. The most rapid H₂S production was seen for Porphyromonas spp and Fusobacterium spp. reaching the maximum color production then the probiotic group and controls.

Further we used H₂S as marker of production of volatile Sulphur compounds in association with oral epithelial cells. The results indicated that when cell were co-cultured with Porphyromonas spp and Fusobacterium spp there was higher production of H₂S whereas when the cells were co-cultured with probiotic cocktail (POC519) and without bacteria there was significantly less production of H₂S (FIG. 5). This results indicated that Porphyromonas and Fusobacterium can utilizes the components of media and epithelial cells to produce H₂S whereas Probiotic cocktail does not produce volatile Sulphur compounds. These data indicate the present probiotic compositions can be used for the prevention and/or treatment of bad breath.

While the present invention has been described through various specific embodiments, routine modification to these embodiments will be apparent to those skilled in the art, which modifications are intended to be included within the scope of this disclosure.

1. A probiotic composition comprising two or more of:
(a) Lactobacillus acidophilus,
(b) L. casei, and/or L. paracasei,
(c) L. reuteri,
(d) Akkermansia muciniphila,
(e) Ruminococcus bromii,
(f) Streptococcus thermophilus and/or Bifidobacterium breve,
(g) Escherichia coli Nissle 1917,
(h) Bifidobacterium lactis, and
one or more classes of phylum Verrucomicrobia and Firmicutes.

2. The probiotic composition of claim 1, wherein a dose of the composition comprises 10⁸-10¹⁰ colony forming units (CFUs) of Lactobacillus acidophilus, 10⁸-10¹⁰ CFUs of L. casei, and/or L. paracasei, 10⁸-10¹⁰ CFUs of L. reuteri, 10⁷-10⁹ CFUs of Akkermansia muciniphila, 10⁷-10⁹ CFUs of Ruminococcus bromii, 10⁶-10⁸ CFUs of Streptococcus thermophilus and/or Bifidobacterium breve, 10⁸-10¹⁰ CFUs of Escherichia coli Nissle 1917, 10⁸-10⁹ CFUs of Bifidobacterium lactis, and 10⁶-10⁷ CFUs of Verrucomicrobia Verrucomicrobiae and/or Firmicutes Bacilli.

3. The probiotic composition of claim 1, wherein the composition is present in a freeze-dried form.

4. A probiotic composition comprising two or more of:
(a) Lactobacillus acidophilus,
(b) L. casei,
(c) L. reuteri,
(d) Akkermansia muciniphila,
(e) Ruminococcus bromii,
(f) Streptococcus salivarius, and
(g) one or more classes of phylum Verrucomicrobia and Firmicutes.

5. The lyophilized probiotic composition of claim 4, wherein the composition comprises 10⁸-10¹⁰ colony forming units (CFUs) of Lactobacillus acidophilus, 10⁸-10¹⁰ CFUs of L. casei, and/or L. paracasei, 10⁸-10¹⁰ CFUs of L. reuteri, 10⁷-10⁹ CFUs of Akkermansia muciniphila, 10⁷-10⁹ CFUs of Ruminococcus bromii, 10⁸-10¹⁰ CFUs of Streptococcus salivarius, and 10⁶-10⁷ CFUs of Verrucomicrobia Verrucomicrobiae and/or Firmicutes bacilli.

6. The probiotic composition of claim 4, wherein the composition is present in a freeze-dried form.

7. A method for improving pancreatic function comprising administering to an individual in need of treatment one or more antibiotics to deplete existing gut microbiome, and administering a probiotic composition of claim 1.

8. The method of claim 7, wherein the gut microbiome is depleted to a level of less than 10⁴ or less than 10³ bacteria.

9. The method of claim 7, wherein the individual is afflicted with pancreatic cancer.

10. The method of claim 9, wherein the pancreatic cancer is pancreatic ductal adenocarcinoma.

11. The method of claim 7, wherein the individual is afflicted with diabetes.

12. The method of claim 11, wherein the diabetes is type 2 diabetes.

13. The method of claim 7, wherein the antibiotic administered is a cocktail of antibiotics comprising two or more of vancomycin, neomycin, metronidazole and amphotericin.

14. The method of claim 7, wherein the probiotic is administered at least 4 weeks after the administration of the antibiotic.

15. The method of claim 14, wherein the probiotic is administered 6-8 weeks after the administration of the antibiotic.

16. A method for treating halitosis comprising administering to the oral cavity of an individual in need of treatment a probiotic composition of claim 4.
